(19) United States
(12) Patent Application Publication
(10) Pub. No.: US 2020/0249214 A1
(43) Pub. Date: Aug. 6, 2020

(54) SYSTEM FOR MONITORING GAS IN CLOSED SPACE AND OPERATING METHOD THEREOF

(71) Applicant: Electronics and Telecommunications Research Institute, Daejeon (KR)

(72) Inventors: Dae Seung YOO, Ulsan (KR); Tae Hyun YOON, Ulsan (KR); Woo Sung JUNG, Ulsan (KR)

(73) Assignee: Electronics and Telecommunications Research Institute, Daejeon (KR)

(21) Appl. No.: 16/684,161

(22) Filed: Nov. 14, 2019

(30) Foreign Application Priority Data
Jan. 31, 2019 (KR) 10-2019-0013154

(51) Int. Cl. G01N 33/00; G06F 17/18

(52) U.S. Cl. CPC G01N 33/0006; G01N 33/0009; G06F 17/18

(57) ABSTRACT

The present invention relates to technology for monitoring a gas in a closed space, and more particularly, to a system for monitoring a gas in a closed space and an operating method of the system, which monitor a gas in a closed space and diagnose and correct an abnormality of a closed-space gas measurement apparatus.

### CROSS-REFERENCE TO RELATED APPLICATIONS

This application claims priority under 35 U.S.C. § 119 to Korean Patent Application No. 10-2019-0013154, filed on Jan. 31, 2019, the disclosure of which is incorporated herein by reference in its entirety.

### TECHNICAL FIELD

The present invention relates to technology for monitoring a gas in a closed space, and more particularly, to a system for monitoring a gas in a closed space and an operating method of the system, which monitor a gas in a closed space and diagnose and correct an abnormality of a closed-space gas measurement apparatus.

### BACKGROUND

Work performed in a closed space completely blocked from the outside like a manufacturing process is very high in risk, and since a noxious gas or an inflammable gas is actually leaked in the closed space, disasters such as suffocation, gas poisoning, firing, and explosion occur or accidents caused by the damages or deaths of workers occurs often.

It is required to continuously measure an inflammable gas and a noxious gas, for performing work in a closed space.

A gas measurement apparatus may be categorized into a portable gas measurement apparatus capable of personally carrying and a stationary gas measurement apparatus fixed at a specific position. In a case where work is performed at a workplace such as a closed space, workers should check safety by measuring a gas before work and then start the work, and moreover, should secure safety by continuously measuring a gas even in work.

Recently, a gas measurement apparatus is connected to a monitoring system provided in a control center over a network and transmits a gas measurement value to the monitoring system, and thus, methods of monitoring a gas in a closed space by using the monitoring system are proposed.

In order to secure the reliability of a gas concentration measured by a gas measurement apparatus, it is required to continuously diagnose and correct a breakdown of the gas measurement apparatus.

However, generally, the maintenance and repair of a gas measurement apparatus are performed by replacing a gas measurement sensor at a certain period and periodically correcting the gas measurement sensor according to a verified correction period.

In a case which uses such a maintenance and repair method performed on a gas measurement apparatus, there is a possibility that workers are exposed at the risk of an accident due to a breakdown of the gas measurement apparatus, and there is a possibility that work efficiency is reduced due to a malfunction of a gas leak alarm.

### SUMMARY

Accordingly, the present invention provides a system for monitoring a gas in a closed space and an operating method of the system, which diagnose and correct an abnormality of a closed-space gas measurement apparatus so as to solve a problem where a gas leak accident occurs and work efficiency decreases due to the breakdown and malfunction of an apparatus for measuring a gas in a closed space.

In one general aspect, an operating method of a gas monitoring system, including a processor and a communication module, for monitoring a gas in a closed space, includes: receiving, by the processor, measurement values, obtained by measuring a gas concentration of the closed space, from a plurality of gas measurement apparatuses by using the communication module; determining, by the processor, a gas measurement apparatus (a correction target) requiring correction by using an average of the measurement values; calculating, by the processor, a compensation value for correcting a measurement value of the determined gas measurement apparatus; transmitting, by the processor, the compensation value to the gas measurement apparatus requiring correction by using the communication module; and receiving, by the processor, a compensated measurement value based on the compensation value from the gas measurement apparatus requiring correction by using the communication module.

In another general aspect, an operating method of a gas monitoring system, including a processor and a communication module, for monitoring a gas in a closed space, includes: receiving, by the processor, measurement values obtained by measuring a gas concentration of the closed space and reliability information, associated with a correction history of each of a plurality of gas measurement apparatuses, from the plurality of gas measurement apparatuses by using the communication module; determining, by the processor, a gas measurement apparatus (a correction target) requiring correction by using the reliability information; calculating, by the processor, a compensation value for correcting a measurement value measured by the gas measurement apparatus determined as the correction target by using a standard deviation of the measurement values and an absolute deviation of the measurement value measured by the gas measurement apparatus determined as the correction target; transmitting, by the processor, the compensation value to the gas measurement apparatus requiring correction by using the communication module; and receiving, by the processor, a compensated measurement value based on the compensation value from the gas measurement apparatus requiring correction by using the communication module.

In another general aspect, an operating method of a gas monitoring system, including a processor and a communication module, for monitoring a gas in a closed space, includes: receiving, by the processor, measurement values, obtained by measuring a gas concentration of the closed space, from a stationary gas measurement apparatus and a portable gas measurement apparatus having reliability equal to or higher than reference reliability by using the communication module; calculating, by the processor, a compensation value for correcting a measurement value measured by the stationary gas measurement apparatus by using an average of the measurement values, a standard deviation of the measurement values, and an absolute deviation of the measurement value measured by the stationary gas measurement apparatus; transmitting, by the processor, the compensation value to the stationary gas measurement apparatus by using the communication module; and receiving, by the processor, a compensated measurement value based on the compensation value from the stationary gas measurement apparatus by using the communication module.

In another general aspect, a gas monitoring system for monitoring a gas in a closed space includes: a communication module configured to receive measurement values, obtained by measuring a gas concentration of the closed space, from a plurality of gas measurement apparatuses; and a processor configured to determine a gas measurement apparatus (a correction target) requiring correction by using an average of the measurement values, calculate a compensation value for correcting a measurement value of the determined gas measurement apparatus, transmit the compensation value to the gas measurement apparatus requiring correction by using the communication module, and receive a compensated measurement value based on the compensation value from the gas measurement apparatus requiring correction by using the communication module.

Other features and aspects will be apparent from the following detailed description, the drawings, and the claims.

### BRIEF DESCRIPTION OF THE DRAWINGS

FIG. 1 is a diagram schematically illustrating an example of a configuration of a system for monitoring a gas in a closed space, according to an embodiment of the present invention.

FIG. 2 is a diagram illustrating an example of a configuration of a gas measurement apparatus according to an embodiment of the present invention.

FIG. 3 is a diagram illustrating an example of a connection relationship based on a data communication network in a closed-space gas monitoring system according to an embodiment of the present invention.

FIG. 4 is a diagram illustrating an example of a configuration of a monitoring system according to an embodiment of the present invention.

FIGS. 5A to 5D are diagrams illustrating examples where a monitoring system according to an embodiment of the present invention groups gas measurement apparatuses.

FIGS. 6A and 6B are diagrams for describing a method of determining and correcting, by a monitoring system according to an embodiment of the present invention, a gas concentration measurement sensor which is to be corrected, based on an absolute deviation and a standard deviation.

FIGS. 7A and 7B are diagrams for describing a method of determining and correcting, by a monitoring system according to an embodiment of the present invention, a gas concentration measurement sensor which is to be corrected, based on an absolute deviation, a standard deviation, and reliability.

FIGS. 8A, 8B and 9 are flowcharts for describing an operation of a closed-space gas monitoring system according to an embodiment of the present invention.

FIG. 10 is a flowchart for describing an operation of a closed-space gas monitoring system according to another embodiment of the present invention.

FIG. 11 is a flowchart for describing a method of diagnosing and correcting a stationary gas measurement apparatus by using a portable gas measurement apparatus according to an embodiment of the present invention.

FIG. 12 is a block diagram illustrating a computing apparatus to which a closed-space gas monitoring system according to another embodiment of the present invention is applied.

### DETAILED DESCRIPTION OF EMBODIMENTS

In embodiments of the present invention disclosed in the detailed description, specific structural or functional descriptions are merely made for the purpose of describing embodiments of the present invention. Embodiments of the present invention may be embodied in various forms, and the present invention should not be construed as being limited to embodiments of the present invention disclosed in the detailed description.

Embodiments of the present invention are provided so that this disclosure will be thorough and complete, and will fully convey the concept of the present invention to one of ordinary skill in the art. Since the present invention may have diverse modified embodiments, preferred embodiments are illustrated in the drawings and are described in the detailed description of the present invention. However, this does not limit the present invention within specific embodiments and it should be understood that the present invention covers all the modifications, equivalents, and replacements within the idea and technical scope of the present invention.

It will be understood that although the terms including an ordinary number such as first or second are used herein to describe various elements, these elements should not be limited by these terms. These terms are only used to distinguish one element from another element. For example, a first element may be referred to as a second element without departing from the spirit and scope of the present invention, and similarly, the second element may also be referred to as the first element.

It will be understood that when an element is referred to as being “connected to” another element, it can be directly connected to the other element or intervening elements may also be present. In contrast, when an element is referred to as being “directly connected to” another element, no intervening elements are present. In addition, unless explicitly described to the contrary, the word “comprise” and variations such as “comprises” or “comprising,” will be understood to imply the inclusion of stated elements but not the exclusion of any other elements. Also, other expressions describing relationships between components such as “˜ between”, “immediately ˜ between” or “adjacent to ˜” and “directly adjacent to ˜” may be construed similarly.

In the following description, the technical terms are used only for explain a specific exemplary embodiment while not limiting the present invention. The terms of a singular form may include plural forms unless referred to the contrary. The meaning of ‘comprise’, ‘include’, or ‘have’ specifies a property, a region, a fixed number, a step, a process, an element and/or a component but does not exclude other properties, regions, fixed numbers, steps, processes, elements and/or components.

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

When some embodiments are differently implementable, a function or an operation described in a specific block may be performed unlike a sequence described in a flowchart. For example, two successive blocks may be substantially simultaneously performed, or may be performed in a reverse sequence depending on a relevant function or operation.

Hereinafter, a closed-space gas monitoring system and an operating method thereof according to an embodiment of the present invention will be described in detail with reference to the accompanying drawings.

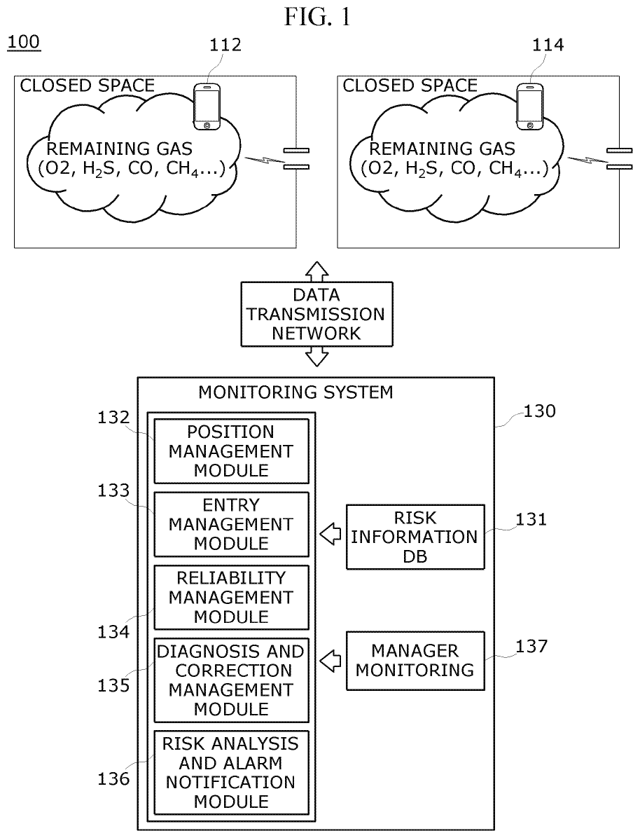

FIG. 1 is a diagram schematically illustrating an example of a configuration of a system 100 for monitoring a gas in a closed space, according to an embodiment of the present invention.

Referring to FIG. 1, the system 100 for monitoring a gas in a closed space according to an embodiment of the present invention may include a plurality of gas measurement apparatuses 112 and 114, a data transmission network (hereinafter referred to as a network) 120, and a monitoring system 130, but a configuration of the system 100 is not limited to an embodiment of the present invention.

The gas measurement apparatuses 112 and 114 may be located in a closed space where work such as a manufacturing process is performed. The gas measurement apparatuses 112 and 114 may measure a gas concentration of the closed space and may transmit the measured gas concentration to the monitoring system 130 over the network 120.

The gas measurement apparatuses 112 and 114 located in the closed space may each be a portable gas measurement apparatus capable of personally carrying and a stationary gas measurement apparatus installed in a closed space.

Therefore, in one closed space, only a portable gas measurement apparatus may be provided, only a stationary gas measurement apparatus may be provided, or a portable gas measurement apparatus and a stationary gas measurement apparatus may be provided.

The gas measurement apparatuses 112 and 114, for example, may each be implemented to measure a concentration of one or more of gases such as oxygen (O₂), carbon monoxide (CO), hydrogen sulfide (H₂S), nitrogen dioxide (NO₂), hydrogen (H₂), and methane (CH₄).

The kinds of gases measurable by the gas measurement apparatuses 112 and 114 are not limited to the above-described gases.

Moreover, the gas measurement apparatuses 112 and 114 may each include a communication module for transmitting a measured gas concentration to the monitoring system 130 over the network 120.

Based on a set environment, the gas measurement apparatuses 112 and 114 may each include one or more of a wired communication module for a connection with a wired network and a wireless communication module for a connection with a wireless network.

The network 120 may be for transmitting data between the gas measurement apparatuses 112 and 114 and the monitoring system 30 and may be a wired network or a wireless network on the basis of a set environment.

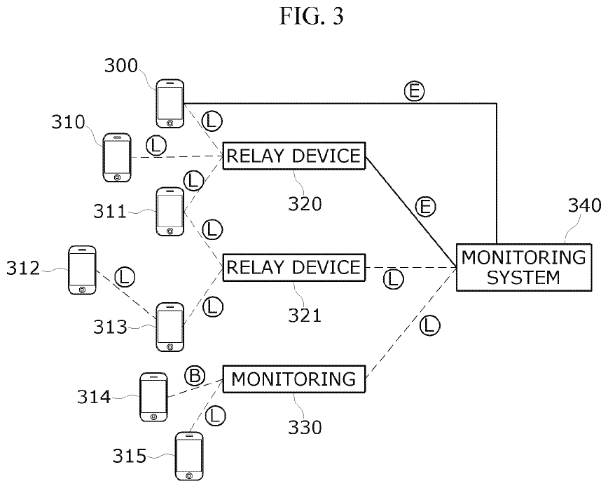

The network 120 may include one or more relay devices (for example, 320 and 321 of FIG. 3) capable of bidirectional communication or a device (for example, 330 of FIG. 3) implemented to perform a function of a relay device.

The monitoring system 130 may be installed in a control center or the like and may diagnose and correct an abnormality of the gas measurement apparatuses 112 and 114 on the basis of a gas concentration from each of the gas measurement apparatuses 112 and 114.

In detail, the monitoring system 130 may include a module 132 which manages workers and positions of the gas measurement apparatuses 112 and 114 on the basis of information collected in real time from a risk information database (DB) 131 and the gas measurement apparatuses 112 and 114, a module 133 which manages entry to a closed space, a module 135 which manages the diagnosis and correction of the gas measurement apparatuses 112 and 114, a module 136 which analyzes a risk situation of the closed space and manages alarm notification based on the risk situation, and a manager monitoring module 137.

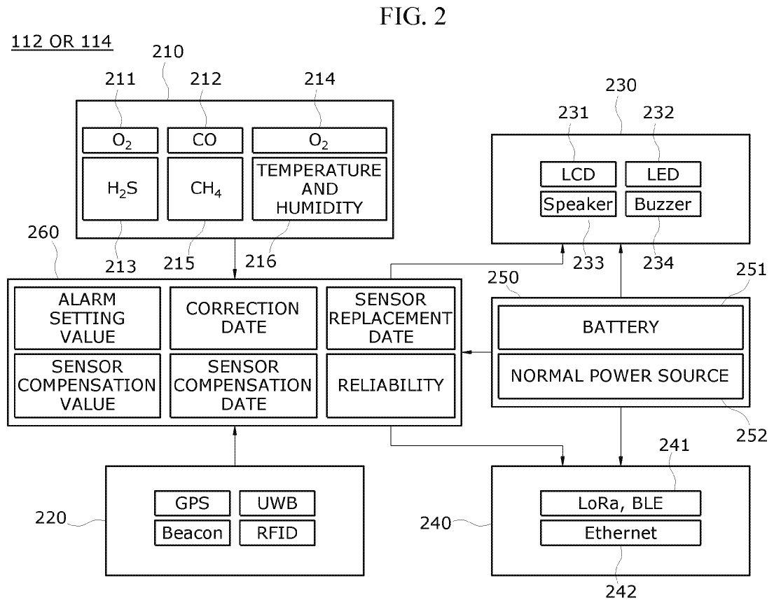

The gas measurement apparatus of the system 100 for monitoring a gas in a closed space according to an embodiment of the present invention will be described below in detail with reference to FIG. 2.

FIG. 2 is a diagram illustrating an example of a configuration of a gas measurement apparatus according to an embodiment of the present invention.

As illustrated in FIG. 2, the gas measurement apparatus 112 or 114 according to an embodiment of the present invention may include a sensor module 210, a position measurement module 220, an interface module 230, a communication module 240, a power module 250, and a control module 260, but a configuration of the gas measurement apparatus 112 or 114 is not limited thereto.

The sensor module 210 may measure a concentration of a gas of a predetermined measurement target in a space (for example, a closed space where a manufacturing process is performed) where the gas measurement apparatus 112 or 114 is located.

Moreover, the sensor module 210 may output information about a measured gas concentration to the control module 260.

For example, the sensor module 210 may be implemented to measure a concentration of one or more of gases such as O₂, CO, H₂S, H₂, and CH₄.

To this end, the sensor module 210 may be configured with various kinds of gas concentration measurement sensors for measuring a concentration of various kinds of gases and may include an oxygen (O₂) sensor 211, a carbon monoxide (CO) sensor 212, a hydrogen sulfide (H₂S) sensor 213, a hydrogen (H₂) sensor 214, and a methane (CH₄) sensor 215.

Since a portable gas measurement apparatus needs a low power design, a sensor included in the portable gas measurement apparatus may have a high sensitivity characteristic.

The sensors 211 to 215 are known to those skilled in the art, and thus, their detailed descriptions are omitted.

For example, the sensor module 210 may be configured with a combination of the oxygen sensor 211, the hydrogen sensor 214, and the methane sensor 215, for measuring a concentration of an inflammable gas.

For example, the sensor module 210 may be configured with a combination of the oxygen sensor 211, the carbon monoxide sensor 212, and the hydrogen sulfide sensor 213, for measuring a concentration of a noxious gas.

For example, the sensor module 210 may be configured with a combination of the oxygen sensor 211, the carbon monoxide sensor 212, the hydrogen sensor 214, and the methane sensor 215 and may overall measure a concentration of an inflammable gas and a concentration of a noxious gas.

In a case where the sensor module 210 includes the hydrogen sensor 214 which is an electrochemical sensor and the methane sensor 215 which is a tactile sensor which is high in amount of consumed energy, the sensor module 210 may first measure a hydrogen concentration, and when the hydrogen concentration is equal to or greater than a certain numerical value, the sensor module 210 may measure a methane concentration, thereby decreasing overall energy consumption.

Furthermore, the sensor module 210 may include a temperature and humidity sensor 216 which is provided as one module for measuring all of a temperature and humidity.

The position measurement module 220 may obtain position information about workers and position information about the gas measurement apparatus 112 or 114.

The position information obtained by the position measurement module 220 may be provided to the monitoring system 130, and for example, may be provided to the monitoring system 130 through the control module 260.

For example, the position measurement module 220 may obtain the position information about the workers and the position information about the gas measurement apparatus 112 or 114 by using global positioning system (GPS), ultra-wide band (UWB), Beacon, radio frequency identification (RFID), and/or the like.

In a case where the position measurement module 220 obtains position information by using GPS or UWB, the position measurement module 220 may measure position coordinates of a worker and a gas measurement apparatus.

In a case where the position measurement module 220 obtains position information by using Beacon or RFID, the position measurement module 220 may obtain space position coordinates of a worker and a gas measurement apparatus.

It is difficult to measure a position by using GPS in an indoor space such as a closed space, and thus, when coordinate information about a position is needed in an indoor space, a position measurement method using UWB technology instead of GPS may be easily performed.

Moreover, in workplaces requiring measurement of a gas concentration, since space position information indicating that being located at a specific workplace such as a closed space is needed rather than coordinate information about a position, Beacon or RFID may be used.

The interface module 230 may visually or acoustically (or vocally) guide various information provided from the outside (for example, the control module, etc.).

For example, the interface module 230 may be configured with display panels such as a liquid crystal display (LCD) 231 and a light emitting diode (LED) 232 and sound output devices such as a speaker 233 and a buzzer 234.

The interface module 230 may output information, included in an accident response message from the monitoring system 130, through the display panel and the sound output device to notify workers of the information.

The communication module 240 may be configured for communication with the monitoring system 130 and may perform uplink communication for transmitting gas concentration information to the monitoring system 130 and downlink communication for receiving information from the monitoring system 130.

Therefore, the communication module 240 may perform bidirectional communication between the gas measurement apparatuses 112 and 114 and the monitoring system 130.

For example, the communication module 240 may be implemented to communicate with the monitoring system 130 by using a wired network or a wireless network or by using all of the wired network and the wireless network.

When the communication module 240 is included in a stationary gas measurement apparatus, the communication module 240 may be implemented to communicate with an Ethernet wired network or a LoRa wireless network.

To this end, the communication module 240 may include a communication module using the Ethernet wired network or a communication module using the LoRa wireless network.

When the communication module 240 is included in a portable gas measurement apparatus, the communication module 240 may be implemented to communicate with a LoRa wireless network or a Bluetooth low energy (BLE) wireless network.

To this end, the communication module 240 may include a communication module using the LoRa wireless network or a communication module using the BLE wireless network.

A wireless communication method applied to a portable gas measurement apparatus according to an embodiment of the present invention may use a method which is low in amount of consumed power, is suitable for a special environment such as a closed space, and is capable of long-distance communication.

For example, a LoRa communication method may have a characteristic of a low power wide area network (LPWAN), may decrease power consumption, may be long in a wireless communication distance capable of arrival at a time, and may support encryption of transmission data, and thus, may be suitable for a portable gas measurement apparatus.

In addition to the LoRa communication method, wireless communication methods (for example, NB-IoT, ZigBee, BLE, etc.) having a feature similar to that of the LoRa communication method may be applied to a portable gas measurement apparatus according to an embodiment of the present invention.

The power module 250 may be configured to supply power to elements (for example, the sensor module 210, the position measurement module 220, the interface module 230, the communication module 240, and the control module 260), requiring power, of the gas measurement apparatus.

For example, the power module 250 may include a battery 251 which is chargeable and dischargeable, and the kind and type of the battery 251 are not limited to one kind and type.

Moreover, the power module 250 may include a normal power source 252 connected to an external power source, and the normal power source 252 may include an external power connector connected to the external power source and an adapter which converts an external power, supplied through the external power connector, into a suitable power and provides the suitable power to an adapter.

When the gas measurement apparatus is a portable gas measurement apparatus, the power module 250 may be implemented to supply a power of the battery 251, and when the gas measurement apparatus is a stationary gas measurement apparatus, the power module 250 may be implemented to supply a power of the normal power source 252.

The control module 260 may control an overall operation of the gas measurement apparatus 100 and may operate with power supplied from the power module 250.

For example, the control module 260 may include at least one processor for performing a function and at least one memory for storing algorithms, programs, and data needed for performing functions and data generated by performing functions.

Moreover, the control module 260 may collect information about a gas concentration measured by the sensor module 210 and may transmit the collected gas concentration information to the monitoring system 130 through the communication module 240.

In this case, the control module 260 may transmit, to the monitoring system 130, the gas concentration information and position information obtained by the position measurement module 220.

Moreover, the control module 260 may exchange messages with the monitoring system 130 to manage unique data of the gas measurement apparatus 10 and control the gas measurement apparatus 10.

When the control module 260 receives an accident response message from the monitoring system 130, the control module 260 may output information, included in the received accident response message, through the interface module 230.

The control module 260 may be configured to store various information, such as information about an alarm setting value, information about a correction date, information about a sensor replacement date, information about a sensor compensation value, information about a sensor compensation date, and information about the reliability of each sensor, in a memory.

Moreover, the control module 260 may transmit, to the monitoring system 130, the gas concentration information and the information about the reliability of each sensor.

The following Table 1 may be a table showing an example of an alarm setting value of each gas.

TABLE 11

| | Alarm setting values | | | |
|---|---|---|---|---|
| Measurement gas | Primary alarm | Secondary alarm | STEL | TWA |
| Oxygen (O₂) | 23.5% vol | 19.5% vol | — | — |
| carbon monoxide | 35 ppm | 100 ppm | 400 ppm | 35 ppm |
| (CO) | | | | |
| hydrogen sulfide | 10 ppm | 15 ppm | 15 ppm | 10 ppm |
| (H₂S) | | | | |
| nitrogen dioxide | 3 ppm | 5 ppm | 5 ppm | 3 ppm |
| (NO₂) | | | | |
| Hydrogen (H₂) | 100 ppm | 500 ppm | — | — |

Here, short term exposure limit (STEL) may be a limit concentration of when a worker is exposed to a harmful factor for 15 minutes at a time and may be a concentration which allows exposure to be performed up to four times for a one-day working time in a case where a one-time exposure interval is one hour or more, under a condition which is equal to or less than the STEL. Also, time weighted average (TWA) may be a concentration calculated by dividing multiplication of occurrence hours and a harmful factor measurement concentration by 8 hours with respect to a daily 8-hour work.

As seen in Table 1, the alarm setting values may be set in units of steps like a primary alarm and a secondary alarm and may be set with respect to STEL and TWA.

Generally, a setting value fixed as the alarm setting value may be used, but in an embodiment of the present invention, the alarm setting value may be updated by the monitoring system 130.

That is, the monitoring system 130 may analyze a risk level based on a working environment, weather, and the kind of work and may transmit an alarm setting value based on the risk level to the gas measurement apparatus 10, thereby allowing the control module 260 to update the alarm setting values.

For example, an alarm setting value of a gas measurement apparatus located near a place at which a welding work is performed, an alarm setting value of a gas measurement apparatus located in a workplace where a closed space is narrow and ventilation is not performed, and an alarm setting value of a gas measurement apparatus which is located at dry weather without wind may be set to be lower than an alarm setting value of a gas measurement apparatus corresponding to a general case.

Gas measurement apparatuses may need the periodic replacement and correction of sensors. On the other hand, in an embodiment of the present invention, the management and maintenance of the gas measurement apparatus may be performed by evaluating the reliability of the gas measurement apparatus on the basis of correction date information, sensor replacement date information, sensor compensation value information, and sensor compensation date information and determining a sensor replacement time or a correction time on the basis of the reliability.

FIG. 3 is a diagram illustrating an example of a connection relationship based on a data communication network in a closed-space gas monitoring system according to an embodiment of the present invention.

In FIG. 3, a gas measurement apparatus 300 may be a stationary gas measurement apparatus, and each of a plurality of gas measurement apparatuses 310 to 315 may be a portable gas measurement apparatus.

A plurality of relay devices 320 and 321 may provide wired/wireless bidirectional communication between the gas measurement apparatuses 300 to 315 and a monitoring system 340.

That is, the relay devices 320 and 321 may provide the monitoring system 340 with information transferred from the gas measurement apparatuses 300 to 315 and may provide the gas measurement apparatuses 300 to 315 with information transferred from the monitoring system 340.

The relay device 320 may be connected to the gas measurement apparatuses 300 to 311 through LoRa wireless communication and may be connected to the monitoring system 340 through Ethernet wired communication. In FIG. 3, LoRa wireless communication is illustrated as a circle where ‘L’ is marked, and the Ethernet wired communication is illustrated as a circle where ‘E’ is marked.

The relay device 321 may be connected to the gas measurement apparatuses 311 to 313 through LoRa wireless communication and may be connected to the monitoring system 340 through LoRa wireless communication.

A monitoring device 330 may be a device which is installed at or near a workplace and receives and displays a gas concentration measured by each of the gas measurement apparatuses 314 and 315 in real time.

Moreover, the monitoring device 330 may be connected to the monitoring system 340 through, for example, LoRa wireless communication and may act as a relay device between the gas measurement apparatuses 314 and 315 and the monitoring system 340.

The gas measurement apparatus 300 may be a stationary gas measurement apparatus and may be connected to the monitoring system 340 through Ethernet wired communication or may be connected to the monitoring system 340 through LoRa wireless communication by using the relay device 320.

The gas measurement apparatus 310 may be a portable gas measurement apparatus and may be connected to the monitoring system 340 by using the relay device 321 which is connected to the gas measurement apparatus 310 through LoRa wireless communication.

The gas measurement apparatus 311 may be a portable gas measurement apparatus and may be connected to the monitoring system 340 by using the relay device 320 and the relay device 321 which are connected to the gas measurement apparatus 311 through LoRa wireless communication.

The gas measurement apparatus 312 may be a portable gas measurement apparatus which is not connected to the relay device 321 through wireless communication, and moreover, may be connected to the relay device 321 through relay communication with the portable gas measurement apparatus 313 and may be connected to the monitoring system 340 by using the relay device 321.

The gas measurement apparatus 313 may be a portable gas measurement apparatus and may be connected to the monitoring system 340 through the relay device 321 which is connected to the gas measurement apparatus 313 through LoRa wireless communication.

The portable gas measurement apparatus 314 may be connected to the monitoring device 330 through BLE wireless communication, and the portable gas measurement apparatus 315 may be connected to the monitoring device 330 through LoRa wireless communication. In FIG. 3, the BLE wireless communication is illustrated as a circle where ‘B’ is marked.

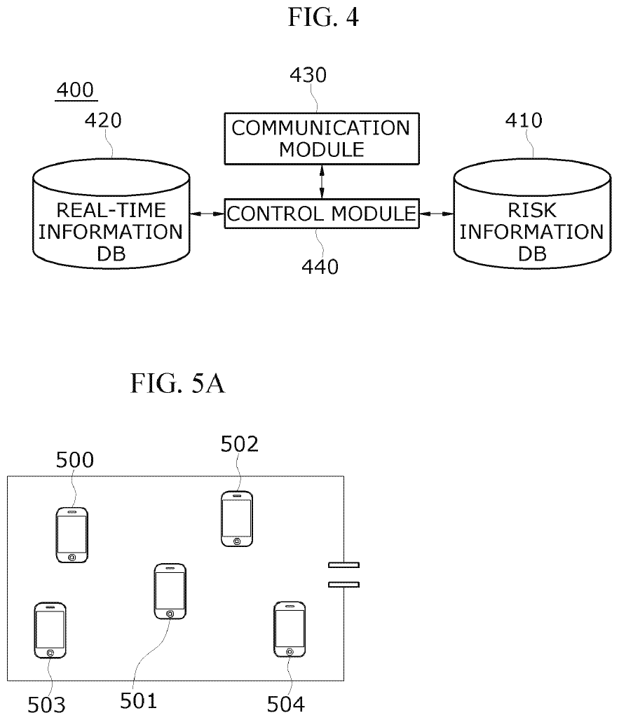

A monitoring system of a system for monitoring a gas in a closed space according to an embodiment of the present invention will be described in detail with reference to FIG. 4.

FIG. 4 is a diagram illustrating an example of a configuration of a monitoring system 400 according to an embodiment of the present invention.

The monitoring system 400 illustrated in FIG. 4 may be applied to the monitoring system 130 of FIG. 1 and may perform a worker/equipment position management function, a closed-zone entry management function, a function of managing reliability of a gas measurement apparatus, a function of diagnosing and correcting a gas measurement apparatus, a risk analysis and measure (warning and evacuation guide) function, and a real-time gas monitoring function on the basis of a risk information database (DB) 410 and a real-time information DB 420.

Particularly, the monitoring system 400 may diagnose the abnormality or not of the gas measurement apparatuses 112 and 114 on the basis of a gas concentration measured by each of the gas measurement apparatuses 112 and 114 and may automatically correct the gas measurement apparatuses 112 and 114 on the basis of a result of the diagnosis.

In this case, the monitoring system 400 may group gas measurement apparatuses located at positions having a similar environment, and on the assumption that gas concentrations measured by gas measurement apparatuses included in the same group are similar, the monitoring system 400 may compare the gas concentrations measured by the gas measurement apparatuses to diagnose and correct the gas measurement apparatuses.

In detail, the monitoring system 400 may include a risk information DB (or a first DB) 410, a real-time collection information DB (or a second DB) 420, a communication module 430, and a control module 440, but a configuration of the monitoring system 400 is not limited to the present embodiment.

The risk information DB 410 may be implemented as at least one storage device (for example, a memory) and may store risk information which is used when the control module 440 is performing a function thereof.

The real-time information DB 420 may be implemented as at least one storage device (for example, a memory) and may store information (for example, gas concentration information from a gas measurement apparatus) provided in real time from the outside.

The communication module 430 may include at least one communication device and may be configured to communicate with an external device (for example, a gas measurement apparatus). Also, the communication module 430 may perform unlink communication for transmitting information from the control module 440 to the gas measurement apparatus 10 and downlink communication for receiving information from the gas measurement apparatus 10.

Therefore, the communication module 430 may perform bidirectional communication between a gas measurement apparatus 112 or 114 and the monitoring system 400.

For example, the communication module 430 may be implemented to communicate with the monitoring system 400 by using a wired network (for example, an Ethernet wired network) or a wireless network (for example, a LoRa wired network) or by using all of the wired network and the wireless network.

The control module 440 may include at least one processor and at least one storage device which stores algorithms, programs, and data needed for performing functions and may perform a predetermined function.

The control module 440 may perform a worker/equipment position management function, a closed-zone entry management function, a function of managing reliability of a gas measurement apparatus, a function of diagnosing and correcting a gas measurement apparatus, a risk analysis and measure (warning and evacuation guide) function, and a real-time gas monitoring function on the basis of the risk information DB 410 and the real-time information DB 420.

The control module 440 may diagnose the abnormality or not of the gas measurement apparatus 10 on the basis of a gas concentration measured by the gas measurement apparatus 10 and position information about the gas measurement apparatus 10 and may automatically correct the gas measurement apparatus 10 on the basis of a result of the diagnosis.

In this case, the control module 440 may group gas measurement apparatuses located at positions having a similar environment, and on the assumption that gas concentrations measured by gas measurement apparatuses included in the same group are similar, the control module 440 may compare the gas concentrations measured by the gas measurement apparatuses to diagnose and correct the gas measurement apparatuses.

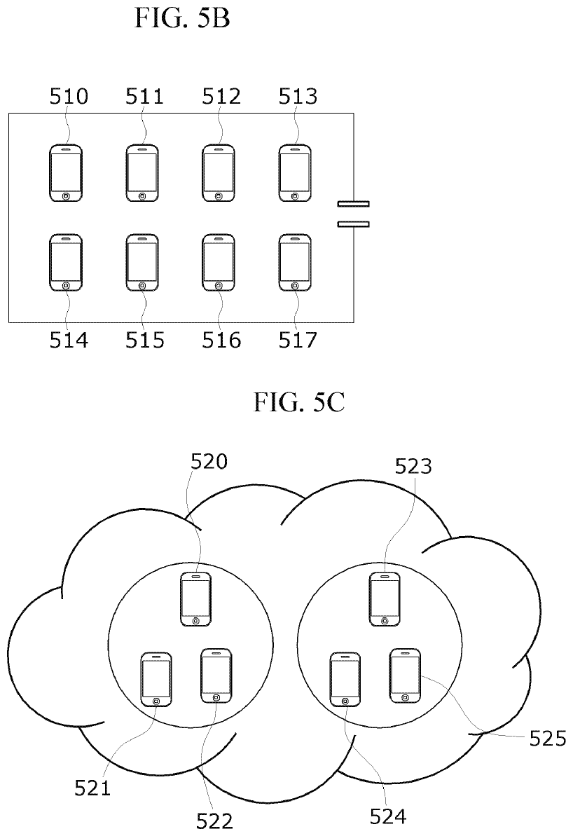

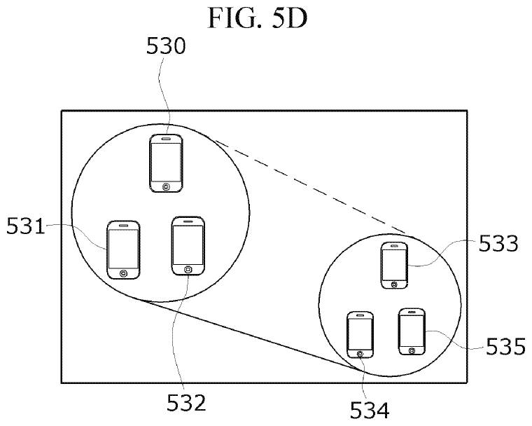

FIGS. 5A to 5D are diagrams illustrating examples where a monitoring system according to an embodiment of the present invention groups gas measurement apparatuses.

FIG. 5A is an example illustrating a plurality of gas measurement apparatuses located in one closed space, and a monitoring system 400 may group a plurality of gas measurement apparatuses 500 to 504, located in one closed space, into one group.

FIG. 5B is an example illustrating a plurality of gas measurement apparatuses located in one space, and a monitoring system 400 may group a plurality of gas measurement apparatuses 510 to 517, located in one space, into one group.

FIG. 5C is an example illustrating a plurality of gas measurement apparatuses located in one open space, and a monitoring system 400 may group a plurality of gas measurement apparatuses 520 to 522 into one group on the basis of positions and may group a plurality of gas measurement apparatuses 523 to 525 into one group on the basis of positions.

FIG. 5D is an example illustrating a case where a plurality of gas measurement apparatuses are located in one work space, and a monitoring system 400 may group a plurality of gas measurement apparatuses 530 to 532, located at an upper portion in a work space, into one group and may group a plurality of gas measurement apparatuses 533 to 535, located at a lower portion in the work space, into one group.

Since a gas is lighter or heavier than air according to a specific gravity, when various kinds of gases are in one space, a gas may be in an upper space or a lower space of a space according to the kind thereof, and thus, gas measurement apparatuses may be grouped based on whether the gas measurement apparatuses are located in an upper space or a lower space of a space.

For example, since the specific gravities of a hydrogen gas and a methane gas among inflammable gases are 0.07 and 0.55 and are lighter than air, the hydrogen gas and the methane gas may be distributed in an upper space of a space, and since the specific gravities of a propane gas and a butane gas are 1.5 and 2.08 and are heavier than air, the propane gas and the butane gas may be distributed in a lower space of the space.

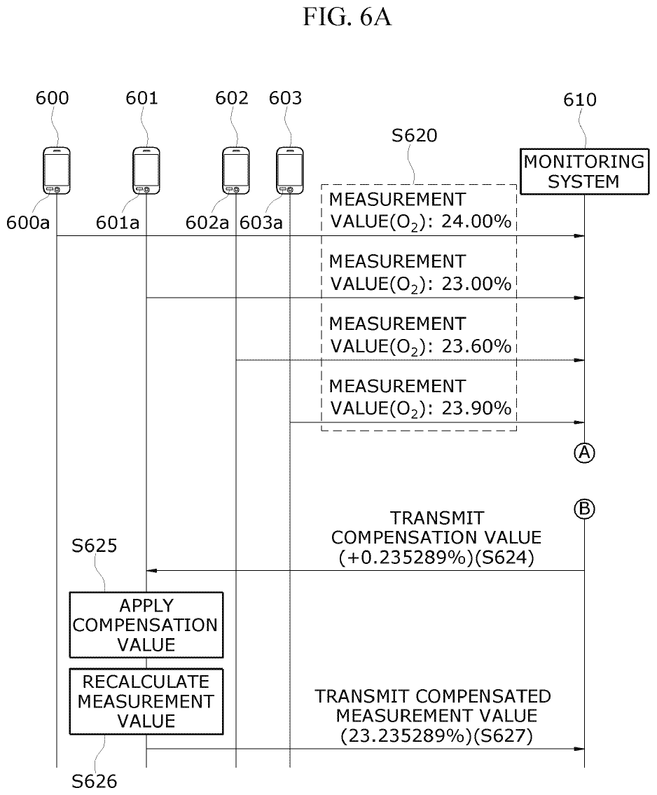

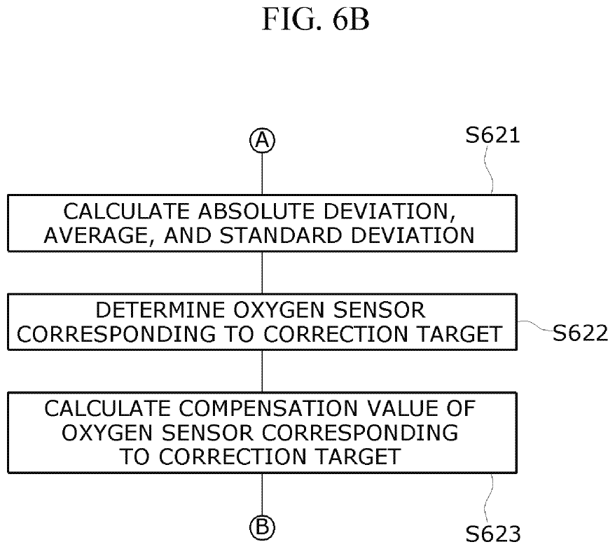

FIGS. 6A and 6B are diagrams for describing a method of determining and correcting, by a monitoring system according to an embodiment of the present invention, a gas concentration measurement sensor which is to be corrected, based on an absolute deviation and a standard deviation.

The following Tables 2 and 3 show values obtained in a process of determining and correcting a gas concentration measurement sensor corresponding to a correction target on the basis of the method illustrated in FIGS. 6A and 6B.

The following Table 2 shows values relevant to oxygen sensors 600a, 601a, 602a, and 603a of four gas measurement apparatuses 600, 601, 602, and 603 and shows an oxygen concentration value (a measurement value), a measurement value absolute deviation, and a sensor compensation value which are measured by each of the oxygen sensors 600a, 601a, 602a, and 603a.

TABLE 2

| | Oxygen | Oxygen | Oxygen | Oxygen |
|---|---|---|---|---|
| | sensor 1 | sensor 2 | sensor 3 | sensor 4 |
| | (600a) | (601a) | (602a) | (603a) |
| Measurement value | 24.00% | 23.00% | 23.60% | 23.90% |
| Measurement value | 0.375 | 0.625 | 0.025 | 0.275 |
| absolute deviation | | | | |
| Sensor compensation | 0 | +0.235289% | 0 | 0 |
| value | | | | |

The following Table 3 shows a before-compensation measurement value average, a before-compensation measurement value standard deviation, an after-sensor-compensation measurement value average, and an after-sensor-compensation measurement value standard deviation which are calculated based on values (measurement values) measured by the four oxygen sensors 600a, 601a, 602a, and 603a.

TABLE 3

| | | | After-sensor- |
|---|---|---|---|
| | | After-sensor- | compensation |
| | Measurement | compensation | measurement |
| Measurement | value | measurement | value |
| value | standard | value | standard |
| average | deviation | average | deviation |
| 23.625% | 0.389711 | 23.684% | 0.297871 |

Hereinafter, a method of determining, by the monitoring system according to an embodiment of the present invention, a gas measurement apparatus requiring correction on the basis of an absolute deviation and a standard deviation will be described with reference to FIGS. 6A and 6B and Tables 2 and 3.

In FIGS. 6A and 6B, it may be assumed that the four gas measurement apparatuses 600 to 603 include the oxygen sensors 600a to 603a and measure an oxygen concentration. Also, it may be assumed that, when an absolute deviation of a measured oxygen concentration is 0.5 or more, a monitoring system 610 is set to perform correction on a corresponding oxygen sensor.

Moreover, it may be assumed that the monitoring system 610 is set to correct an oxygen sensor corresponding to a correction target so that an absolute deviation of the oxygen sensor corresponding to the correction target converges to a standard deviation of measurement values. Also, a method of determining and correcting a sensor requiring correction may be variously set based on a policy. For example, an absolute deviation criterion is set to a value lower than a predetermined value so as to perform diagnosis and correction at a higher level. Also, a reference absolute deviation may be differently set based on the kind of a measured gas, the kind of a sensor, and a measurement range of the sensor.

Referring again to FIGS. 6A and 6B and Tables 2 and 3, in step S620, the monitoring system 610 may receive oxygen concentration measurement values “24.00%, 23.00%, 23.60%, and 23.90%”, measured by the oxygen sensors 600a to 603a, from the four gas measurement apparatuses 600 to 603.

In step S621, the monitoring system 610 may calculate an average (a measurement value average) of the measurement values and may calculate an absolute deviation of each of the measurement values and a measurement value standard deviation of the measurement values by using the measurement value average.

Subsequently to step S621, in step S622, the monitoring system 610 may determine an oxygen sensor corresponding to a correction target.

In step S622, the monitoring system 610 may compare the measurement value absolute deviation with a reference absolute deviation and may determine, as a correction-target oxygen sensor, an oxygen sensor having a condition where the measurement value absolute deviation is equal to or greater than the reference absolute deviation.

In Table 2, a measurement value absolute deviation relevant to the oxygen sensor 1 600a, a measurement value absolute deviation relevant to the oxygen sensor 3 602a, and a measurement value absolute deviation relevant to the oxygen sensor 4 603a are less than 0.5 which is the reference absolute deviation, but a measurement value absolute deviation relevant to the oxygen sensor 2 601a is equal to or greater than 0.5, whereby the oxygen sensor 2 601a is a correction target.

Therefore, in step S622, the monitoring system 610 may determine the oxygen sensor 2 601a as a correction target.

Subsequently to step S622, in step S623, the monitoring system 610 may calculate a compensation value for compensating for the oxygen sensor 2 601a.

In step S623, based on a predetermined program, the monitoring system 610 may calculate a compensation value which enables a measurement value absolute deviation, associated with the correction-target oxygen sensor, to be the measurement value standard deviation.

In step S623, based on the predetermined program, the monitoring system 610 may calculate a compensation value which enables a measurement value, associated with the correction-target oxygen sensor, to be the measurement value average.

In an embodiment of the present invention, since the monitoring system 610 is set to calculate a sensor compensation value which enables the measurement value absolute deviation to be the measurement value standard deviation, the monitoring system 610 may calculate a sensor compensation value as +0.235289 in step S623. That is, the sensor compensation value may be calculated from a difference between the measurement value absolute deviation and the measurement value standard deviation.

Moreover, since each of the oxygen sensor 1 600a, the oxygen sensor 3 602a, and the oxygen sensor 4 603a is not a correction target, a compensation value of each of the oxygen sensor 1 600a, the oxygen sensor 3 602a, and the oxygen sensor 4 603a may be 0.

Subsequently to step S623, the monitoring system 610 may transmit the compensation value to the gas measurement apparatus 2 601 in step S624, and thus, may apply the compensation value in step S625, thereby correcting the oxygen sensor 2 601a.

In step S625, the monitoring system 610 may transmit the compensation value to a control module of the gas measurement apparatus 2 601, and the control module of the gas measurement apparatus 2 601 may apply the compensation value to a measurement value of the oxygen sensor 2 601a in step S625 to recalculate a measurement value in step S626 and may transmit a compensated measurement value to the monitoring system 610 in step S627.

As seen in Table 3, as a measurement value of the oxygen sensor 2 is compensated for, the measurement value standard deviation decreases from 0.389711 to 0.297871. A decrease in standard deviation may denote that oxygen concentrations measured by the four oxygen sensors 600a to 603a become similar through correction of the oxygen sensor 2 601a.

As described above, according to an embodiment of the present invention, determination of a correction-target sensor and calculation and application of a compensation value may be automatically performed based on a gas concentration measured by a gas measurement apparatus.

FIGS. 6A and 6B and Tables 2 and 3 are an embodiment where a compensation value suitable for a correction-target sensor is capable of being calculated based on an absolute deviation and a standard deviation.

However, a case where a compensation value suitable for a correction-target gas concentration sensor fails to be calculated may occur.

Hereinafter, a method of correcting a correction-target gas concentration measurement sensor by additionally using the reliability of the gas concentration measurement sensor will be described as a method capable of being applied to a case where a measurement value of a gas concentration measurement sensor should be compensated for but the correction-target gas concentration measurement sensor is not corrected based on an absolute deviation and a standard deviation.

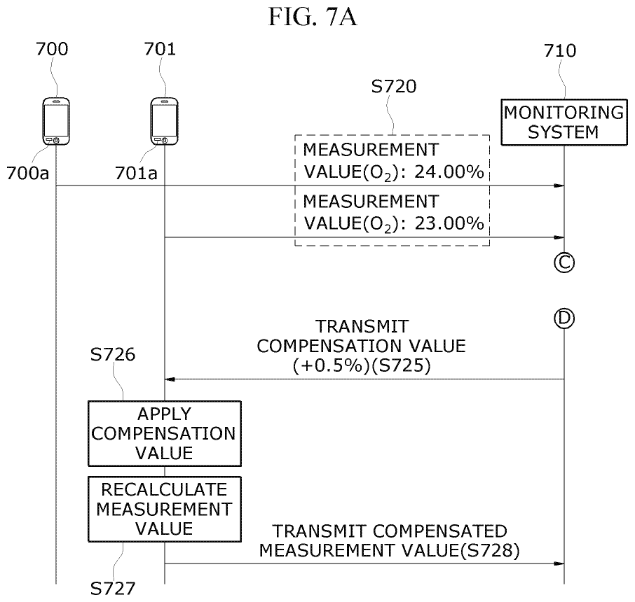

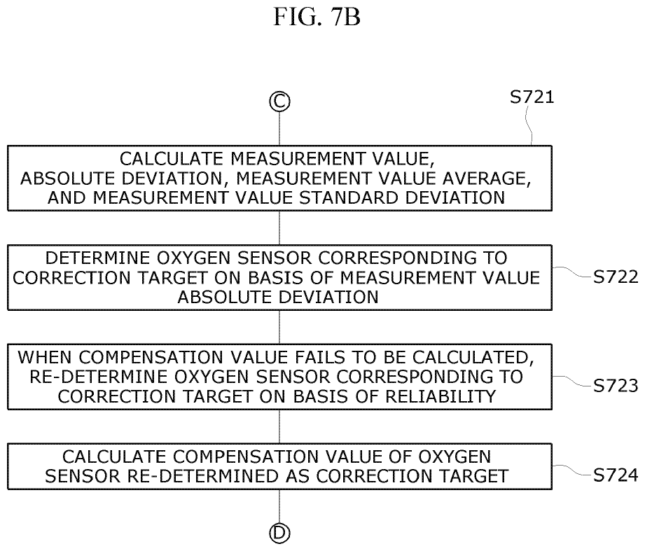

FIGS. 7A and 7B are diagrams for describing a method of determining and correcting, by a monitoring system according to an embodiment of the present invention, a gas concentration measurement sensor which is to be corrected, based on an absolute deviation, a standard deviation, and reliability.

The following Tables 4 and 5 show values obtained in a process of determining and correcting a gas concentration measurement sensor corresponding to a correction target according to the illustration of FIGS. 7A and 7B.

The following Table 4 shows values relevant to oxygen sensors 700a and 701a of two gas measurement apparatuses 700 and 701 included in the same group and shows an oxygen concentration value (a measurement value) measured by each of the oxygen sensors 700a and 701a, a measurement value absolute deviation, a sensor compensation value, and reliability.

TABLE 4

| | Oxygen | Oxygen |
|---|---|---|
| | sensor 5 | sensor 6 |
| | (700a) | (701a) |
| Measurement value | 24.00% | 23.00% |
| Measurement value | 0.5 | 0.5 |
| absolute deviation | | |
| Sensor compensation | 0 | +0.5% |
| value | | |
| Reliability | 95 | 85 |

The following Table 5 shows a before-compensation measurement value average, a before-compensation measurement value standard deviation, an after-compensation measurement value average, and an after-compensation measurement value standard deviation which are calculated based on values (measurement values) measured by the two oxygen sensors 700a and 701a.

TABLE 5

| | | | After-sensor- |
|---|---|---|---|
| | | After-sensor- | compensation |
| | Measurement | compensation | measurement |
| Measurement | value | measurement | value |
| value | standard | value | standard |
| average | deviation | average | deviation |
| 23.5% | 0.5 | 23.75% | 0.25 |

Hereinafter, a method of determining and correcting, by the monitoring system according to an embodiment of the present invention, a correction-target gas concentration measurement sensor on the basis of reliability will be described with reference to FIGS. 7A and 7B and Tables 4 and 5.

In FIGS. 7A and 7B, it may be assumed that the two gas measurement apparatuses 700 and 701 include the oxygen sensors 700a and 701a and measure an oxygen concentration. Also, it may be assumed that, when an absolute deviation of a measured oxygen concentration is 0.5 or more, a monitoring system 710 is set to perform correction on a corresponding oxygen sensor.

Moreover, it may be assumed that, when a gas concentration measurement sensor corresponding to a correction target is not determined and corrected based on a measurement value absolute deviation and a measurement value standard deviation, the monitoring system 710 is set to determine a low-reliability gas concentration measurement sensor as a correction target.

Moreover, it may be assumed that, in a case which determines and corrects a gas concentration measurement sensor corresponding to a correction target on the basis of reliability, the monitoring system 710 is set to calculate a compensation value in order for a current measurement value standard deviation to have a half value.

A method of determining and correcting a gas concentration measurement sensor corresponding to a correction target may be variously set according to a policy. For example, an absolute deviation criterion may be set to a value lower than a predetermined value so as to perform diagnosis and correction at a higher level, and a measurement value of a correction-target sensor may be set to calculate a compensation value which converges to a measurement value average. Also, a reference absolute deviation may be differently set based on the kind of a measured gas, the kind of a sensor, and a measurement range of the sensor.

Referring again to FIGS. 7A and 7B and Tables 4 and 5, in step S720, the monitoring system 710 may receive oxygen concentration measurement values “24.00% and 23.00%”, measured by the oxygen sensors 700a and 701a, from the two gas measurement apparatuses 700 and 701.

In step S721, the monitoring system 710 may calculate an absolute deviation of each of the measurement values, an average (a measurement value average) of the measurement values, and a measurement value standard deviation.

In step S722, the monitoring system 710 may compare the measurement value absolute deviation with a reference absolute deviation and may determine, as an oxygen sensor corresponding to a correction target, an oxygen sensor having a condition where the measurement value absolute deviation is equal to or greater than the reference absolute deviation.

In Table 4, a measurement value absolute deviation relevant to the oxygen sensor 5 700a may be 0.5 and a measurement value absolute deviation relevant to the oxygen sensor 6 701a may be 0.5, and thus, the oxygen sensor 5 700a and the oxygen sensor 6 701a may each be a correction target. Therefore, in step S722, the monitoring system 710 may determine an oxygen sensor corresponding to a correction target on the basis of a measurement value absolute deviation.

Subsequently to step S722, the monitoring system 710 should calculate a compensation value so that a measurement value absolute deviation relevant to the oxygen sensor corresponding to the correction target is a measurement value standard deviation, but as seen in Tables 4 and 5, since a measurement value absolute deviation is the same as a measurement value standard deviation, a compensation value fails to be calculated.

As described above, when a compensation value of an oxygen sensor corresponding to a correction target is not calculated based on an absolute deviation and a standard deviation, the monitoring system 710 may again determine a correction target on the basis of the reliability of the oxygen sensor corresponding to the correction target in step S723.

In step S723, the monitoring system 710 may determine a low-reliability oxygen sensor as a correction target, and thus, may determine the oxygen sensor 6 701a as a correction target.

In step S723, the monitoring system 710 may request reliability information about each oxygen sensor from each gas measurement apparatus and may be provided with the reliability information.

Subsequently to step S723, the monitoring system 710 may calculate a compensation value of an oxygen sensor re-determined as a correction target in step S724.

In step S724, the monitoring system 710 may calculate a compensation value which enables a measurement value standard deviation to have a half value, based on a predetermined program.

Therefore, the monitoring system 710 may calculate +0.5 as a compensation value of the oxygen sensor 6 701a corresponding to a correction target so that a measurement value standard deviation is changed from 0.5 to 0.25.

Subsequently to step S724, the monitoring system 710 may transmit the compensation value to the gas measurement apparatus 6 701 in step S725, and thus, may apply the compensation value in step S726, thereby correcting the oxygen sensor 6 701a of the gas measurement apparatus 6 701.

Moreover, a control module of the gas measurement apparatus 6 701 may apply the compensation value to a measurement value of the oxygen sensor 6 701a in step S726 to recalculate a measurement value in step S727 and may transmit a compensated measurement value to the monitoring system 710 in step S728.

As seen in Table 5, as a measurement value of the oxygen sensor 6 701a is compensated for, the measurement value standard deviation decreases from 0.5 to 0.25. A decrease in standard deviation may denote that oxygen concentrations measured by oxygen sensors become similar through correction of the oxygen sensor 6 701a.

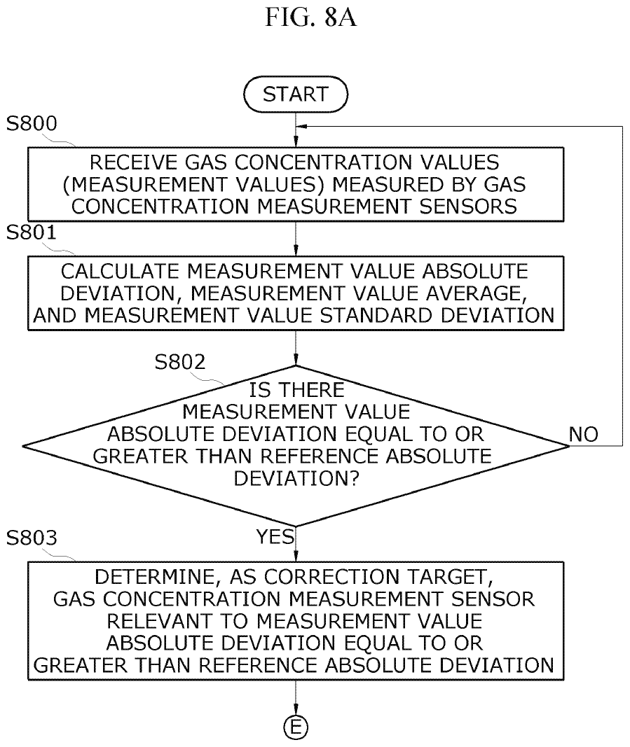

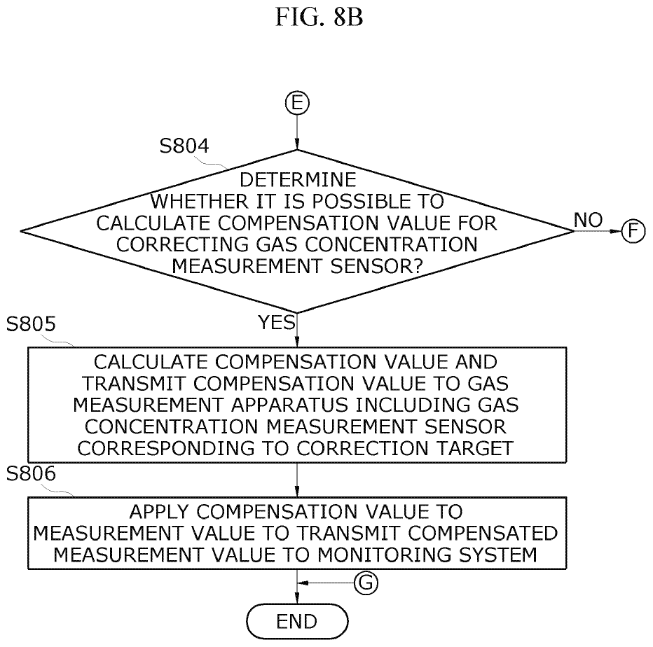

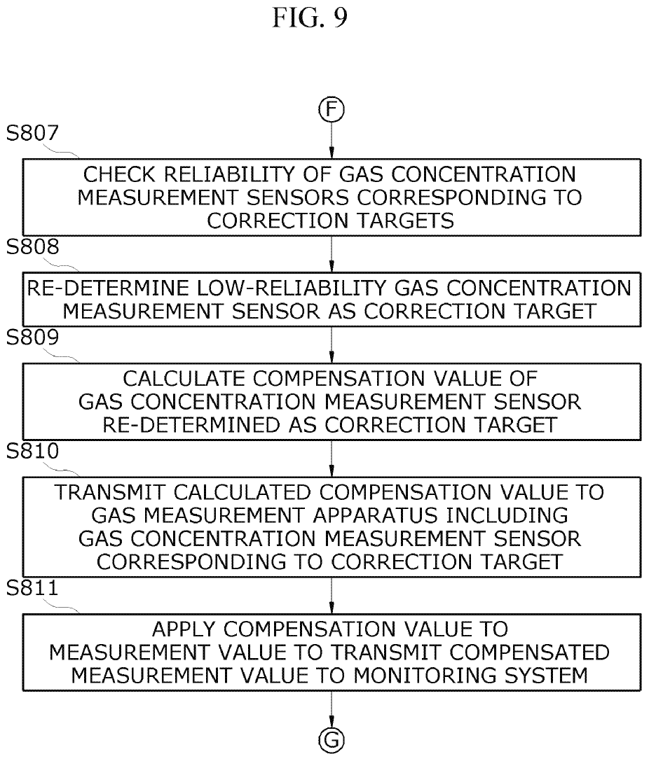

FIGS. 8A, 8B and 9 are flowcharts for describing an operation of a closed-space gas monitoring system according to an embodiment of the present invention.

A step-based operation illustrated in FIGS. 8A and 8B may be performed by the closed-space gas monitoring system described above with reference to FIGS. 1 to 7B, and it may be assumed that a plurality of gas measurement apparatuses including a gas concentration measurement sensor for measuring the same gas concentration is located at one closed workplace.

First, in step S800, a monitoring system may receive a gas concentration value (hereinafter referred to as a measurement value), measured by a gas concentration measurement sensor included in each of a plurality of gas measurement apparatuses, over a predetermined network (120 of FIG. 1).

Subsequently to step S800, the monitoring system may calculate an absolute deviation (a measurement value absolute deviation) of each of the measurement values, an average (a measurement value average) of the measurement values, and a standard deviation (a measurement value standard deviation) of the measurement values in step S801.

Subsequently to step S801, the monitoring system may compare the measurement value absolute deviation with a reference absolute deviation to determine whether there is a measurement value absolute deviation which is equal to or greater than the reference absolute deviation in step S802.

When it is determined that there is no measurement value absolute deviation which is equal to or greater than the reference absolute deviation in step S802, the monitoring system may end an operation or may stand by until next measurement values are received, based on a predetermined program.

When it is determined that there is the measurement value absolute deviation which is equal to or greater than the reference absolute deviation in step S802, the monitoring system may determine, as a correction target, a gas concentration measurement sensor relevant to the measurement value absolute deviation which is equal to or greater than the reference absolute deviation in step S803.

Subsequently to step S803, the monitoring system may determine whether it is possible to calculate a compensation value for correcting a gas concentration measurement sensor corresponding to a correction target in step S804.

In step S804, the monitoring system may determine whether it is possible to calculate a compensation value which enables a measurement value absolute deviation, associated with the gas concentration measurement sensor corresponding to the correction target, to be a measurement value standard deviation. For example, when the measurement value absolute deviation differs from the measurement value standard deviation, the monitoring system may determine that it is possible to calculate the compensation value, and when the measurement value absolute deviation differs from the measurement value standard deviation, the monitoring system may determine that it is unable to calculate the compensation value.

In step S804, the monitoring system may determine whether it is possible to calculate a compensation value which enables a measurement value, associated with the gas concentration measurement sensor corresponding to the correction target, to be a measurement value average. For example, when the measurement value differs from the measurement value average, the monitoring system may determine that it is possible to calculate the compensation value, and when the measurement value differs from the measurement value average, the monitoring system may determine that it is unable to calculate the compensation value.

When it is determined that it is possible to calculate the compensation value for correcting the gas concentration measurement sensor corresponding to the correction target in step S804, the monitoring system may calculate the compensation value and may transmit the compensation value to a gas measurement apparatus including the gas concentration measurement sensor corresponding to the correction target in step S805.

In step S805, the monitoring system may calculate the compensation value which enables the measurement value absolute deviation, associated with the gas concentration measurement sensor corresponding to the correction target, to be the measurement value standard deviation. For example, the compensation value may be calculated from a difference between the measurement value absolute deviation and the measurement value standard deviation.

In step S805, the monitoring system may calculate a compensation value which enables a measurement value, associated with the gas concentration measurement sensor corresponding to the correction target, to be a measurement value average. For example, the compensation value may be calculated from a difference between the measurement value and the measurement value average.

Subsequently to step S805, a gas measurement apparatus which has received the compensation value may apply the compensation value to the measurement value to transmit a compensated measurement value to the monitoring system in step S806. Here, the compensation value may be applied to the measurement value on the basis of various operational formulas.

Subsequently to step S806, the monitoring system may apply the compensated measurement value to recalculate a measurement value average and a measurement value standard deviation.

When it is determined that it is unable to calculate the compensation value for correcting the gas concentration measurement sensor corresponding to the correction target in step S804, as illustrated in FIG. 9, the monitoring system may check reliability of gas concentration measurement sensors which are determined as correction targets in step S803, in step S807.

Subsequently to step S807, the monitoring system may re-determine a low-reliability gas concentration measurement sensor as a correction target in step S808 and may calculate a compensation value of the gas concentration measurement sensor re-determined as the correction target in step S809.

In step S809, the monitoring system may calculate a compensation value which enables the measurement value standard deviation, calculated in step S801, to have a half value.

Subsequently to step S809, the monitoring system may transmit the calculated compensation value to a gas measurement apparatus including a gas concentration measurement sensor corresponding to a correction target in step S810.

Subsequently to step S810, a gas measurement apparatus which has received the compensation value may apply the compensation value to the measurement value to transmit a compensated measurement value to the monitoring system in step S811.

Subsequently to step S811, the monitoring system may apply the compensated measurement value to recalculate a measurement value average and a measurement value standard deviation.

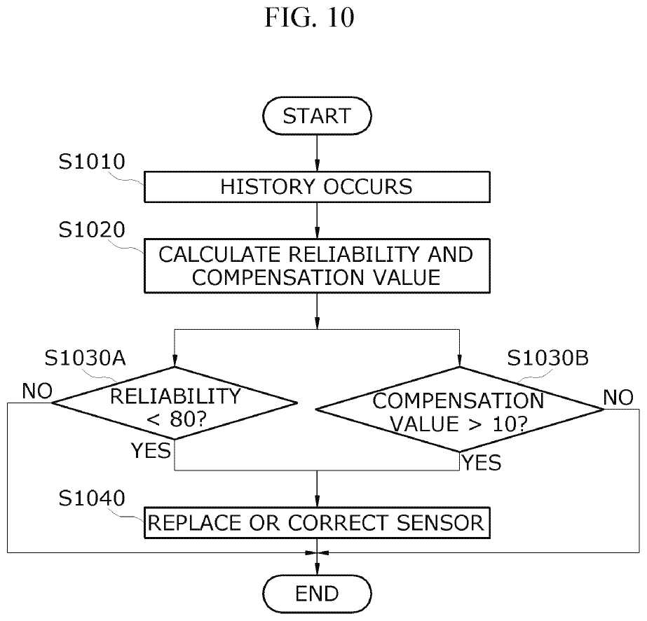

FIG. 10 is a flowchart for describing an operation of a closed-space gas monitoring system according to another embodiment of the present invention.

Referring to FIG. 10, as a history (including a correction event and a replacement event) of a gas concentration measurement sensor occurs, the closed-space gas monitoring system according to another embodiment of the present invention may calculate the reliability and a compensation value of the gas concentration measurement sensor and may perform a process of determining a sensor replacement timing and a sensor correction timing on the basis of the calculated reliability and compensation value.

First, in step S1010, a process of checking, by a monitoring system, the occurrence of history data of a specific gas concentration measurement sensor may be performed. Here, for example, the history data may be as shown in the following Table 6.

TABLE 6

| | | | Add/subtract | Compensation |
|---|---|---|---|---|
| Division | History | Reliability | point | value |
| 1 | Initial set reliability | 95 | 0 | 0 |
| 2 | Convergence of | 96 | +1 | 0 |
| | measurement value | | | |
| | to measurement | | | |
| | value average | | | |
| 3 | Convergence of | 97 | +1 | 0 |
| | measurement value | | | |
| | to measurement | | | |
| | value average | | | |
| 4 | Elapse of 6 months | 96.5 | −0.5 | 0 |
| | after replacing sensor | | | |
| 5 | Elapse of 1 month after | 96 | −0.5 | 0 |
| | correction | | | |
| 6 | Elapse of 7 months | 95.5 | −0.5 | 0 |
| | after replacing sensor | | | |
| 7 | Elapse of 2 months | 95 | −0.5 | 0 |
| | after correction | | | |
| 8 | Automatic correction | 94 | −1 | +0.2 |
| 9 | Automatic correction | 93 | −1 | +0.4 |
| 10 | Elapse of 8 months | 92.5 | −0.5 | +0.4 |
| | after replacing sensor | | | |
| 11 | Elapse of 3 months | 92 | −0.5 | +0.4 |
| | after correction | | | |
| 12 | Automatic correction | 90 | −2 | +0.8 |
| 13 | Automatic correction | 88 | −2 | +1.3 |

As shown in Table 6, history data may be generated when a specific event occurs in a gas concentration measurement sensor. The specific event may include an event where a measurement value obtained through measurement of a gas concentration by the gas concentration measurement sensor converges to a measurement value average, an event where a used duration of a replaced gas concentration measurement sensor is greater than a specific duration (for example, 6 months, 7 months, or 8 months), an event where a used duration of a compensation-value-corrected gas concentration measurement sensor is greater than a specific duration (for example, 1 months, 2 months, or 3 months), and an event where a compensation value of a gas concentration measurement sensor is automatically corrected based on the method described above with reference to FIGS. 8A, 8B and 9. Subsequently, in step S1020, when history data is generated, a process of calculating reliability and a compensation value on the basis of the generated history data may be performed.

Calculation of reliability may be a method of increasing or decreasing reliability by a predetermined point on the basis of the kind of an event defined in history data and the number of occurrence of the event.

For example, as shown in Table 6, whenever an event where a measurement value converges to a measurement value average, initial set reliability “95” may increase by +1 each time. Also, whenever an event where a used duration of a replaced gas concentration measurement sensor is greater than a specific duration occurs or an event where a used duration of a compensation-value-corrected gas concentration measurement sensor is greater than a specific duration occurs, current reliability may decrease by −0.5 each time. Also, as the number of automatic correction of a compensation value increases, current reliability may decrease by −1 or −2 each time.

Similarly, calculation of a compensation value may be a method of increasing or decreasing reliability by a predetermined point on the basis of the kind of an event defined in history data and the number of occurrence of the event.

For example, a compensation value may increase by 0.2, 0.4, 0.8, or 1.3 each time on the basis of the number of automatic correction of a compensation value, an event where a used duration of a replaced gas concentration measurement sensor is greater than a specific duration, and an event where a used duration of a compensation-value-corrected gas concentration measurement sensor is greater than a specific duration.

Subsequently, a process of comparing reference reliability with the reliability which is calculated in step S1020 may be performed in step S1030A, and simultaneously, a process of comparing a reference compensation value with the compensation value which is calculated in step S1020 may be performed in step S1030B.

The reference reliability and the reference compensation value may be previously defined for determining a sensor replacement or correction timing. In an embodiment of the present invention, the reference reliability may be 80, and the reference compensation value may be 10.

Subsequently, when the reliability calculated in step S1020 is lower than the reference reliability or the compensation value calculated in step S1020 is greater than the reference compensation value, a corresponding gas concentration measurement sensor may be determined as a replacement target or a correction target in step 1040.

For example, when the reliability calculated in step S1020 is equal to or higher than the reference reliability or the compensation value calculated in step S1020 is equal to or less than the reference compensation value, a corresponding gas concentration measurement sensor may be determined as a sensor which does not need replacement or correction currently, and all processes may end without replacement or correction.

As described above, the monitoring system according to an embodiment of the present invention may determine a replacement timing and a correction timing on the basis of the reliability and a compensation value of a gas concentration measurement sensor. Therefore, the monitoring system according to an embodiment of the present invention may not simply replace or correct a gas concentration measurement sensor according to a predetermined replacement or correction timing but may calculate the reliability and a compensation value of a gas concentration measurement sensor whenever specific history data occurs and may determine a replacement timing and a correction timing of the gas concentration measurement sensor on the basis of the calculated reliability and compensation value, thereby solving a problem where the management and maintenance of a gas concentration measurement sensor are not smoothly performed by reason of that a replacement timing or a correction timing does not arrive, despite a breakdown of the gas concentration measurement sensor.

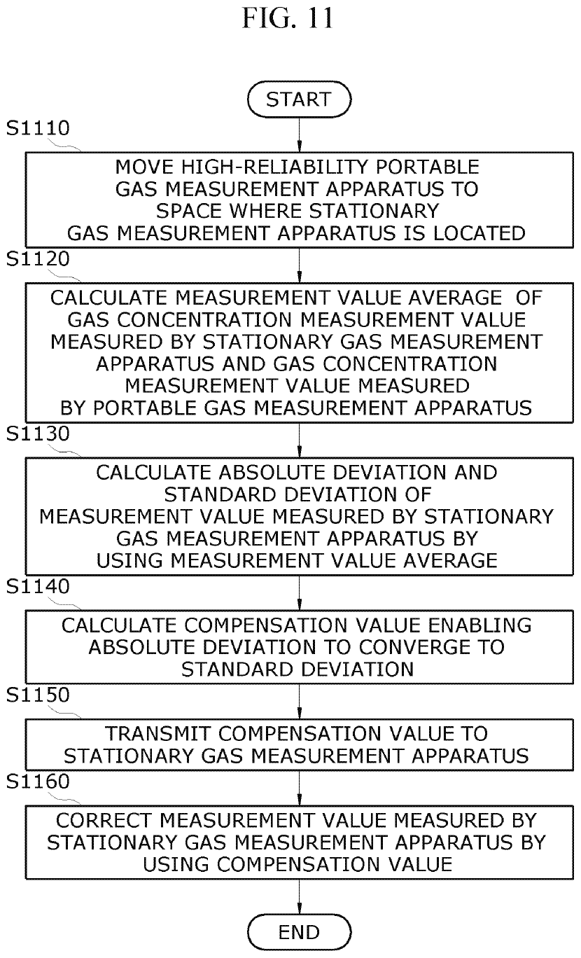

FIG. 11 is a flowchart for describing a method of diagnosing and correcting a stationary gas measurement apparatus by using a portable gas measurement apparatus according to an embodiment of the present invention.

A portable gas measurement apparatus may be high in possibility that the portable gas measurement apparatus is located in the same space along with a plurality of gas measurement apparatuses, but since a stationary gas measurement apparatus is fixed, the portable gas measurement apparatus may be low in possibility that the portable gas measurement apparatus is located in the same space along with a plurality of gas measurement apparatuses. In this case, the method described above with reference to FIGS. 7 to 12 may not be applied to a gas concentration measurement sensor included in the stationary gas measurement apparatus, and thus, it may be impossible to diagnose and correct the gas concentration measurement sensor included in the stationary gas measurement apparatus. That is, when only a stationary gas measurement apparatus is located and another stationary gas measurement apparatus or a portable gas measurement apparatus is not located in an arbitrary space, an absolute deviation and a standard deviation may not be calculated, and thus, a correction target may not be determined and a compensation value may not be calculated.

In order to solve such a problem, in an embodiment of the present invention, a method of diagnosing and correcting a stationary gas measurement apparatus by using a high-reliability portable gas measurement apparatus will be described below.

Referring to FIG. 11, in step S1110, a process of moving a high-reliability portable gas measurement apparatus to a space where a stationary gas measurement apparatus is located may be performed. Here, the high-reliability gas measurement apparatus may be an apparatus having reliability which is equal to or higher than the reference reliability. The reference reliability may be, for example, 95.

Subsequently, in step S1120, a monitoring system may calculate an average (a measurement value average) of a gas concentration measurement value measured by a stationary gas measurement apparatus and a gas concentration measurement value measured by a portable gas measurement apparatus.

Subsequently, in step S1130, the monitoring system may calculate an absolute deviation and a standard deviation of a measurement value measured by the stationary gas measurement apparatus by using the measurement value average. In this case, an absolute deviation and a standard deviation of a measurement value measured by the portable gas measurement apparatus may not need to be calculated. This is because the stationary gas measurement apparatus and a grouped portable gas measurement apparatus are high in reliability, and thus, a high-reliability portable gas measurement apparatus does not need to be calculated, whereby an absolute deviation and a standard deviation each needed for correction do not need to be calculated. In other words, in the present embodiment, a process of determining a correction target may be omitted.

Subsequently, in step S1140, a process of calculating a compensation value enabling an absolute deviation to converge to a standard deviation may be performed by the monitoring system. Here, for example, the compensation value may be calculated from a difference between the absolute deviation and the standard deviation.

Subsequently, in step S1150, a process of transmitting the compensation value, calculated in step S1130, to the stationary gas measurement apparatus may be performed by the monitoring system.

Subsequently, in step S1160, a process of correcting the measurement value measured by the stationary gas measurement apparatus by using the compensation value may be performed. Subsequently, a corrected measurement value may be transmitted to the monitoring system again.

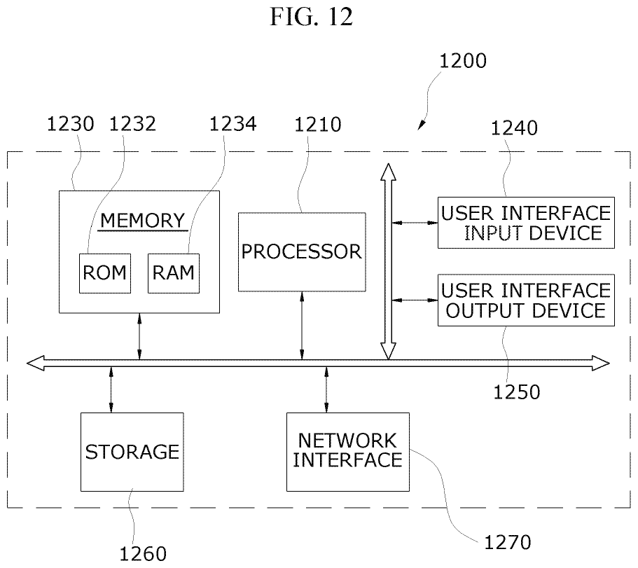

FIG. 12 is a block diagram illustrating a computing apparatus 1200 to which a closed-space gas monitoring system according to another embodiment of the present invention is applied.

Referring to FIG. 12, the computing system 1200 may include at least one processor 1210, a memory 1230, a user interface input device 1240, a user interface output device 1250, a storage 1260, and a network interface 1270, which are connected to one another through a bus 1220.

The processor 1210 may be a semiconductor device for executing instructions stored in a central processing unit (CPU) or the memory 1230 and/or the storage 1260.

The processor 1210 may perform a process of calculating a measurement value average of measurement values measured by gas measurement apparatuses located in the same space and an absolute deviation and a standard deviation of each of the measurement values.

Moreover, the processor 1210 may update the reliability of each gas measurement apparatus on the basis of a use history (a correction history and a replacement history). Also, the processor 1210 may store updated reliability in the memory 1230 or the storage 1260 and may control an operation of the network interface 1270 to transmit the updated reliability to a corresponding gas measurement apparatus.

Each of the memory 1230 and the storage 1260 may include various kinds of volatile or non-volatile storage mediums. For example, the memory 1230 may include read only memory (ROM) and random access memory (RAM).

A method or a step of an algorithm described above in association with embodiments disclosed in the present specification may be directly implemented with hardware, a software module, or a combination thereof, which is executed by the processor 1210.

The software module may be provided in RAM, flash memory, ROM, erasable programmable read only memory (EPROM), electrical erasable programmable read only memory (EEPROM), a register, a hard disk, an attachable/detachable disk, or a storage medium (i.e., the memory 1230 and/or the storage 1260) such as CD-ROM.

An exemplary storage medium may be coupled to the processor 1210, and the processor 1210 may read out information from the storage medium and may write information in the storage medium. In other embodiments, the storage medium may be provided as one body with the processor 1210. The processor and the storage medium may be provided in application specific integrated circuit (ASIC). The ASIC may be provided in a user terminal. In other embodiments, the processor and the storage medium may be provided as individual components in a user terminal.

Exemplary methods according to embodiments may be expressed as a series of operation for clarity of description, but such a step does not limit a sequence in which operations are performed. Depending on the case, steps may be performed simultaneously or in different sequences. In order to implement a method according to embodiments, a disclosed step may additionally include another step, include steps other than some steps, or include another additional step other than some steps.

Various embodiments of the present disclosure do not list all available combinations but are for describing a representative aspect of the present disclosure, and descriptions of various embodiments may be applied independently or may be applied through a combination of two or more.

Moreover, various embodiments of the present disclosure may be implemented with hardware, firmware, software, or a combination thereof. In a case where various embodiments of the present disclosure are implemented with hardware, various embodiments of the present disclosure may be implemented with one or more application specific integrated circuits (ASICs), digital signal processors (DSPs), digital signal processing devices (DSPDs), programmable logic devices (PLDs), field programmable gate arrays (FPGAs), general processors, controllers, microcontrollers, or microprocessors.

The scope of the present disclosure may include software or machine-executable instructions (for example, an operation system (OS), applications, firmware, programs, etc.), which enable operations of a method according to various embodiments to be executed in a device or a computer, and a non-transitory computer-readable medium capable of being executed in a device or a computer each storing the software or the instructions.

According to the embodiments of the present invention, technology for monitoring a gas in a closed space and diagnosing and correcting an abnormality of a closed-space gas measurement apparatus may be provided.

By using technology for monitoring a gas in a closed space according to the embodiments of the present invention, a gas concentration value measured by a gas measurement apparatus provided in a closed space of a workplace may be transmitted to the monitoring system, and the monitoring system may monitor a collected gas concentration value in real time, overall check and compare a position of the gas measurement apparatus and a measured gas concentration value to diagnose the abnormality or not of the gas measurement apparatus, and calculate and apply a compensation value to correct a sensor.

Moreover, since an operation of diagnosing the abnormality or not of a gas measurement apparatus and correcting a sensor is automatically performed, a problem where a gas leak accident occurs due to the use of a broken-down gas measurement apparatus and work efficiency decreases due to a malfunction of a gas leak alarm may be solved, and time and cost taken in diagnosing a gas measurement apparatus and correcting a sensor may be considerably reduced.

Hereinabove, the system for monitoring a gas in a closed space and the operating method of the system according to the present invention have been described according to the embodiments, but the scope of the present invention is not limited to a specific embodiment. The present invention may be corrected and modified within the technical scope obvious to those skilled in the art.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An operating method of a gas monitoring system, including a processor and a communication module, for monitoring a gas in a closed space, the operating method comprising:
receiving, by the processor, measurement values, obtained by measuring a gas concentration of the closed space, from a plurality of gas measurement apparatuses by using the communication module;
determining, by the processor, a gas measurement apparatus (a correction target) requiring correction by using an average of the measurement values;
calculating, by the processor, a compensation value for correcting a measurement value of the determined gas measurement apparatus;
transmitting, by the processor, the compensation value to the gas measurement apparatus requiring correction by using the communication module; and
receiving, by the processor, a compensated measurement value based on the compensation value from the gas measurement apparatus requiring correction by using the communication module.

2. The operating method of claim 1, wherein the determining of the gas measurement apparatus requiring correction comprises:
calculating an absolute deviation of a measurement value measured by each of the plurality of gas measurement apparatuses by using the average of the measurement values;
comparing the absolute deviation with a predefined reference absolute deviation; and
determining the gas measurement apparatus requiring correction by using a comparison result of the comparing of the absolute deviation with the predefined reference absolute deviation.

3. The operating method of claim 2, wherein the determining of the gas measurement apparatus requiring correction by using the comparison result comprises determining a gas measurement apparatus, which has transmitted a measurement value representing an absolute deviation equal to or greater than the predefined reference absolute deviation, as the gas measurement apparatus requiring correction.

4. The operating method of claim 2, wherein the predefined reference absolute deviation is set based on a kind of a gas to be measured, a kind of a gas concentration measurement sensor installed in the gas measurement apparatus, and a measurement range of the gas concentration measurement sensor.

5. The operating method of claim 1, wherein the calculating of the compensation value comprises calculating, as the compensation value, a difference between a measurement value measured by a gas measurement apparatus determined as the correction target and the average of the measurement values.

6. The operating method of claim 1, wherein the calculating of the compensation value comprises calculating, as the compensation value, a difference between an absolute deviation of a measurement value measured by a gas measurement apparatus determined as the correction target and a standard deviation of the measurement values.

7. An operating method of a gas monitoring system, including a processor and a communication module, for monitoring a gas in a closed space, the operating method comprising:
receiving, by the processor, measurement values obtained by measuring a gas concentration of the closed space and reliability information, associated with a correction history of each of a plurality of gas measurement apparatuses, from the plurality of gas measurement apparatuses by using the communication module;
determining, by the processor, a gas measurement apparatus (a correction target) requiring correction by using the reliability information;
calculating, by the processor, a compensation value for correcting a measurement value measured by the gas measurement apparatus determined as the correction target by using a standard deviation of the measurement values and an absolute deviation of the measurement value measured by the gas measurement apparatus determined as the correction target;
transmitting, by the processor, the compensation value to the gas measurement apparatus requiring correction by using the communication module; and
receiving, by the processor, a compensated measurement value based on the compensation value from the gas measurement apparatus requiring correction by using the communication module.

8. The operating method of claim 7, wherein the reliability information comprises a reliability point added or subtracted based on number of corrections of the gas measurement apparatus, a time elapsing from a time at which the gas measurement apparatus is corrected, and a time elapsing from a time at which the gas measurement apparatus is replaced.

9. The operating method of claim 7, wherein the determining of the gas measurement apparatus requiring correction comprises determining a gas measurement apparatus, having a lowest reliability point, as a correction target on the basis of the reliability information.

10. The operating method of claim 7, wherein the calculating of the compensation value comprises calculating the compensation value from a difference between the absolute deviation and the standard deviation.

11. An operating method of a gas monitoring system, including a processor and a communication module, for monitoring a gas in a closed space, the operating method comprising:
receiving, by the processor, measurement values, obtained by measuring a gas concentration of the closed space, from a stationary gas measurement apparatus and a portable gas measurement apparatus having reliability equal to or higher than reference reliability by using the communication module;
calculating, by the processor, a compensation value for correcting a measurement value measured by the stationary gas measurement apparatus by using an average of the measurement values, a standard deviation of the measurement values, and an absolute deviation of the measurement value measured by the stationary gas measurement apparatus;
transmitting, by the processor, the compensation value to the stationary gas measurement apparatus by using the communication module; and
receiving, by the processor, a compensated measurement value based on the compensation value from the stationary gas measurement apparatus by using the communication module.

12. The operating method of claim 11, wherein the portable gas measurement apparatus having reliability equal to or higher than the reference reliability is an apparatus requiring no correction of a measurement value.

13. The operating method of claim 11, wherein the calculating of the compensation value comprises calculating, as the compensation value, a difference between an absolute deviation of a measurement value measured by the stationary gas measurement apparatus and a standard deviation of the measurement values.

14. A gas monitoring system for monitoring a gas in a closed space, the gas monitoring system comprising:
a communication module configured to receive measurement values, obtained by measuring a gas concentration of the closed space, from a plurality of gas measurement apparatuses; and
a processor configured to determine a gas measurement apparatus (a correction target) requiring correction by using an average of the measurement values, calculate a compensation value for correcting a measurement value of the determined gas measurement apparatus, transmit the compensation value to the gas measurement apparatus requiring correction by using the communication module, and receive a compensated measurement value based on the compensation value from the gas measurement apparatus requiring correction by using the communication module.

15. The gas monitoring system of claim 14, wherein the processor performs a process of calculating an absolute deviation of a measurement value measured by each of the plurality of gas measurement apparatuses by using the average of the measurement values, a process of comparing the absolute deviation with a predefined reference absolute deviation, and a process of determining the gas measurement apparatus requiring correction by using a comparison result of the comparing of the absolute deviation with the predefined reference absolute deviation.

16. The gas monitoring system of claim 15, wherein the processor performs a process of determining a gas measurement apparatus, which has transmitted a measurement value representing an absolute deviation equal to or greater than the predefined reference absolute deviation, as the gas measurement apparatus requiring correction.

17. The gas monitoring system of claim 14, wherein the processor performs a process of calculating, as the compensation value, a difference between a measurement value measured by a gas measurement apparatus determined as the correction target and the average of the measurement values.

18. The gas monitoring system of claim 14, wherein the processor performs a process of calculating, as the compensation value, a difference between an absolute deviation of a measurement value measured by a gas measurement apparatus determined as the correction target and a standard deviation of the measurement values.
